(19) United States
(12) Patent Application Publication
(10) Pub. No.: US 2026/0069395 A1
(43) Pub. Date: Mar. 12, 2026

(54) Prosthetic Sling Anchor and Surgical Implant System

(71) Applicants: Rebecca U. Ruefer, Payson, AZ (US); Beda B.G. Ruefer, Payson, AZ (US); Peter Barthold, Balingen (DE)

(72) Inventors: Rebecca U. Ruefer, Payson, AZ (US); Beda B.G. Ruefer, Payson, AZ (US); Peter Barthold, Balingen (DE)

(21) Appl. No.: 19/320,927

(22) Filed: Sep. 5, 2025

Related U.S. Application Data
(60) Provisional application No. 63/691,881, filed on Sep. 6, 2024.

(51) Int. Cl. A61F 2/00; A61L 31/04

(52) U.S. Cl. CPC A61F 2/0045; A61L 31/048; A61F 2220/0016

(57) ABSTRACT

An anchor for receiving an end of an article for use as a surgical implant includes a base portion having one or more apertures to receive the end of the article such that the article is secured to the anchor; and a head portion attached to the base portion and extending therefrom, the head portion having a tip and at least two wings extending downward from the tip such that each of the at least two wings end in a wing tip; the head portion is for insertion through bodily tissue via the tip and is to be retained within the bodily tissue via the at least two wings; and the base portion is to retain the article to the bodily tissue.

### RELATED APPLICATIONS

This application claims priority to U.S. Provisional Application No. 63/691,881, filed Sep. 6, 2024, which is incorporated by reference in its entirety herein.

### BACKGROUND OF THE INVENTION

### 1. Field

The disclosed embodiments relate generally to the field of prosthetic medical devices. More specifically, the field relates to the development of prosthetic medical slings implanted for supporting internal body structures, including anchors configured to attach said medical slings to bodily tissue.

### 2. Description of the Related Art

Pubovaginal sling procedures are very prevalently used to offer support needed to stabilize a patient's urethra or bladder. The most common device used in executing such a process is an elongated flexible strip constructed of a nonabsorbable polypropylene mesh material, the ends of which can be anchored elsewhere in the patient's body, and support is offered to prevent incontinence.

### SUMMARY

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages will be apparent from the following detailed description of the embodiments and the accompanying drawing figures.

Embodiments of the present invention include an anchor for receiving an end of an article configured for use as a surgical implant, the anchor comprising a base portion having one or more apertures configured to receive the end of the article such that the article is secured to the anchor; and a head portion attached to the base portion and extending therefrom, the head portion having a tip and at least two wings extending downward from the tip such that each of the at least two wings end in a wing tip; wherein the head portion is configured for insertion through bodily tissue via the tip and is configured to be retained within the bodily tissue via the at least two wings; and wherein the base portion is configured to retain the article to the bodily tissue.

Other embodiments of the present invention include a surgical implant system, comprising an article configured to support a bodily tissue and an anchor configured to secure the article to and retain the article to the bodily tissue. The article having a body extending from a first end forming a first connection location to a second end forming a second connection location, the body formed of a biocompatible, bacteria-resistant fluoropolymer material; wherein the biocompatible, bacteria-resistant fluoropolymer material comprises a plurality of nodes and a plurality of fibrils, the plurality of fibrils interconnecting the plurality of nodes to form a plurality of pores; and wherein the biocompatible, bacteria-resistant fluoropolymer material comprises a microporous structure which is relatively closed to ingrowth and bacterial penetration. The anchor having a base portion having one or more apertures configured to connect to the first connection location of the article such that the article is secured to the anchor; and a head portion attached to the base portion and extending therefrom, the head portion having a tip and at least two wings extending downward from the tip such that each of the at least two wings end in a wing tip; wherein the head portion is configured for insertion through the bodily tissue via the tip and is configured to be retained within the bodily tissue via the at least two wings; and wherein the base portion is configured to retain the article to the bodily tissue.

### BRIEF DESCRIPTION OF THE SEVERAL VIEWS OF THE DRAWINGS

Illustrative embodiments are described in detail below with reference to the attached drawing figures, which are incorporated by reference herein and wherein:

FIG. 1A shows an overall view for a first embodiment of an article in accordance with the present invention.

FIG. 1B shows a cross-sectional view taken from line 1B-1B in FIG. 1A.

FIG. 2 is a micrograph taken of a material suitable for the article of FIG. 1 and manufactured according to the process steps expressed herein where a multiaxial expansion is executed.

FIG. 3 a micrograph taken at a cross section of the material shown in FIG. 2.

FIG. 4 is a first side view of the article of FIG. 1.

FIG. 5A is a perspective view of a first embodiment of an anchor for use with the article of FIG. 4.

FIG. 5B is a top view of the anchor of FIG. 5A.

FIG. 5C is a side view of the anchor of FIG. 5A.

FIG. 6A is a top view showing two anchors as depicted in FIG. 5A attached to the article of FIG. 4.

FIG. 6B is a bottom view showing two anchors as depicted in FIG. 5A attached to the article of FIG. 4.

FIG. 7A is a top view showing two anchors as depicted in FIG. 5A after being bent and attached to the article of FIG. 4.

FIG. 7B is a bottom view showing two anchors as depicted in FIG. 5A after being bent and attached to the article of FIG. 4.

FIG. 8A is side view of two anchors as depicted in FIG. 5A after being bent and attached to the article of FIG. 4.

FIG. 8B is another side view of two anchors as depicted in FIG. 5A after being bent and attached to the article of FIG. 4.

FIG. 9A is a top perspective view of a second embodiment of an anchor for use with the article of FIG. 4.

FIG. 9B is a bottom perspective view of the anchor of FIG. 9A.

FIG. 10 is a top view of the anchor of FIG. 9A.

FIG. 11 is a side view of the anchor of FIG. 9A.

FIG. 12 is a cross-sectional view taken from line 12-12 of FIG. 10.

FIG. 13 is an end view of the anchor of FIG. 9A.

FIG. 14 is a top view showing two anchors as depicted in FIG. 9A attached to the article of FIG. 4.

FIG. 15 is a bottom view showing two anchors as depicted in FIG. 9A attached to the article of FIG. 4.

The drawing figures do not limit the invention to the specific embodiments disclosed and described herein. The drawings are not necessarily to scale, emphasis instead being placed upon clearly illustrating the principles of the invention.

### DETAILED DESCRIPTION

The following detailed description references the accompanying drawings that illustrate specific embodiments in which the invention can be practiced. The embodiments are intended to describe aspects of the invention in sufficient detail to enable those skilled in the art to practice the invention. Other embodiments can be utilized and changes can be made without departing from the scope of the invention. The following detailed description is, therefore, not to be taken in a limiting sense. The scope of the invention is defined only by the appended claims, along with the full scope of equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments but is not necessarily included. Thus, the technology can include a variety of combinations and/or integrations of the embodiments described herein.

Synthetic slings have been available for implant procedures since the early 2000's. The most common form of sling is constructed of polypropylene, which is typically comprised of woven or knit filaments. A polypropylene sling presents open structure which allows bacteria to penetrate. This can lead to post-operative infection. Although these devices have been implanted in many thousands of patients, there remain many post-implantation problems that have yet to be resolved. Most of these problems are clinically significant, and can end with surgical retrieval of the devices, which can lead to internal tissue damage.

One problem with these prior art polypropylene mesh slings is damage that can be caused upon surgical implant. Those skilled in the art will recognize that during surgery the polypropylene mesh sling is worked through bodily tissues, e.g., being pushed or pulled therethrough using needles or other devices. The edges of these prior art slings may be rough, and when passed through the body during surgical implant can create tissue damage, e.g., scar tissue, etc.

Any limited damage created upon implant of the polypropylene sling is not necessarily considered a bad thing by artisans in the field, in that the damage causes an immediate inflammatory response, which ultimately helps incorporate the device structurally. But this damage can compromise tissues in undesirable ways also.

Another problem is that the polypropylene mesh degrades over time. This degradation can result in migration of sling segments or filaments, or even extrusion of the same. These obvious failures create harm, and often result in the need for removal of an already-implanted sling by a surgery far more complicated and potentially harmful than the initial implant procedure, and can leave behind massive scar tissue, as well as result in chronic pain moving forward.

Another problem is that of infection. The vulnerability of the polypropylene to infection is due in large part to the nature of the mesh material. Following implantation of a conventional polypropylene mesh sling, it is considered desirable that the surrounding tissues grow into the device. This ingrowth is seen as necessary to secure the polypropylene device in place. But this also makes the device extremely difficult to remove surgically, e.g., in the case of complications such as infection or structural degradation over time. Many times, surgery to remove the sling creates serious problems to the tissues on or around the implant.

Prior art devices, e.g., polypropylene tapes, have been anchored using suturing or polypropylene anchors. Conventionally, these polypropylene anchors are formed into a hook design, wherein the hook is configured for securing to bodily tissue. The use of a hook design has drawbacks, such as difficulty inserting through bodily tissue.

The invention disclosed herein relates to a prosthetic medical sling made of a novel synthetic material, in embodiments, a relatively closed expanded Polytetrafluoroethylene (ePTFE) structure. The invention further includes an anchor configured for anchoring said sling to bodily tissue as a surgical implant. The inventive design of the medical device provides for a strong, supportive, biocompatible sling that resists bacterial invasion and disintegration. Embodiments of the synthetic prosthetic medical sling are configured to resist bacterial infection, exhibit significant long-term strength to support tissues, and avoid the development of scar tissue. The device also will not deteriorate over time like the conventional systems. Further, the sling embodiments exhibit strength adequate for intended use, are supple and tissue compliant to minimize scar tissue formation, and are durable, as PTFE, a fluoropolymer, is known to be resistant to biodegradation. The fully PTFE supporting system provides optimal internal mechanics required for adequate long-term support, while avoiding internal damage and maintaining biocompatibility. Additionally, the all-PTFE system allows for material stability through the manufacturing process as opposed to conventional polypropylene products.

Embodiments are comprised of a processed fluoropolymer, such as Polytetrafluoroethylene (PTFE). The article includes a band portion comprised of expanded PTFE (“ePTFE”) which is formed by expansion under heat. The ePTFE band can be manufactured to have fibril lengths such that the article is a relatively closed structure to prevent the infiltration of bacteria, yet supple for tissue compliance, and adequately strong for its intended use as a tissue support device. And of course, PTFE is fully biocompatible and will not degrade.

In terms of the band microporous structure, the ePTFE article is, in embodiments, characterized by a plurality of nodes that are connected together by a plurality of fibrils. The nodes are essentially solid PTFE, having a density of between about 2.0 grams to about 2.2 grams per cubic centimeter, whereas the density of the expanded material is less than about 2.0 grams per cubic centimeter. The shape, size, and orientation of the nodes and fibrils within the structure can be controlled by varying the expansion rate, expansion ratio, number of expansion axes, and other processing parameters to yield many different structures. It is also known that properties such as the expandability and microstructure of the expanded article vary with the molecular weight, particle size, and other physical characteristics of the PTFE resin.

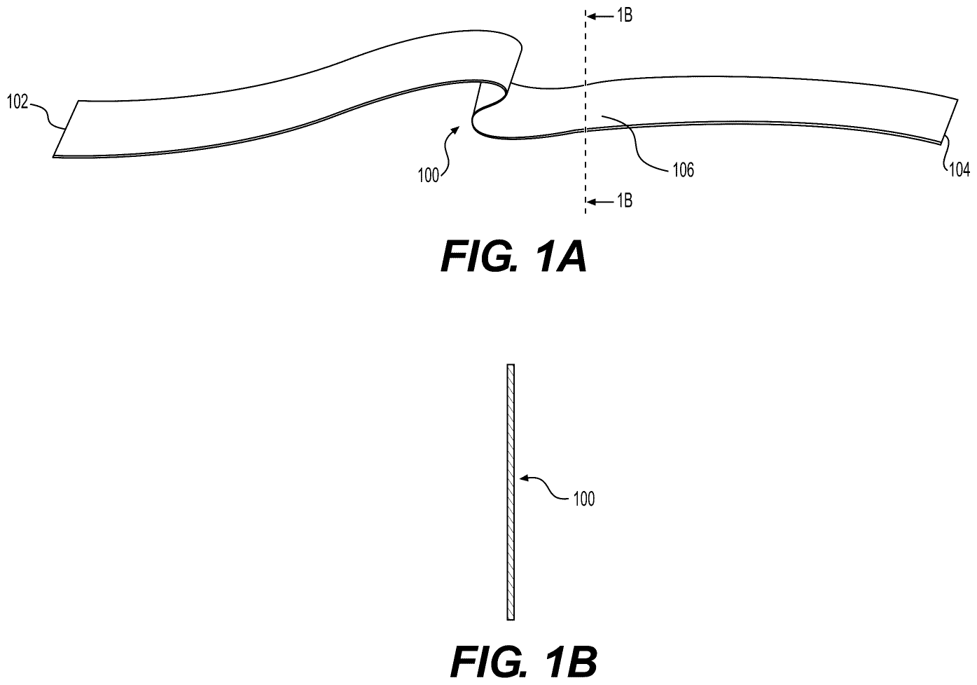

An embodiment of the ePTFE band, which is the supporting portion of the device, along with opposing substantially solid PTFE anchors is useable as an implant device (e.g., sling). The supporting portion is configured as a flat (roughly/substantially planar) elongated sheet 100 (as seen in FIGS. 1A and 1B before connection of the anchors).

The ePTFE portion 100 disclosed has shown no ingrowth as well as preventing bacterial penetration. Thus, the article avoids the well-known infection problems existing in the polypropylene prior art devices. Additionally, because ingrowth is avoided, surgical removal is relatively easy to accomplish, if necessary, and leaves little, if any, scar tissue or other damage.

The embodiment 100 for the supporting article is shown in perspective in FIG. 1A and is shown in cross section in 1B (which is taken at Section 1B-1B in the figure in FIG. 1A). The article 100 will be usable as a surgical supporting device and includes a first end 102, a second end 104, and a substantially flat elongated body 106 (configured of a fluoropolymer in embodiments). The substantially flat elongated body 106 of the first version 100 is, in embodiments, comprised of multiaxially expanded PTFE that will ultimately reflect a node/fibril structure that can be made according to the practices discussed hereinafter. In some embodiments, the substantially flat elongated body 106 is comprised of uniaxially expanded PTFE, that again reflects a node/fibril structure according to the practices discussed hereinafter.

The article is made, in embodiments, by expanding PTFE in either one direction (i.e. uniaxially) or alternatively in multiple dimensions (i.e., multiaxially). Beforehand, a resin paste can be mixed with an extrusion-aid such as mineral spirits, and then that paste can be compressed at relatively low pressures into a pre-extrusion form, e.g., as a pellet.

In embodiments, the material is extruded as a substantially flat article.

The PTFE article is then calendared while wet to a desired thickness. Because the article will be partially wet with mineral spirits, the process then moves on to a drying step where the lubricant is removed by subjecting it to a temperature slightly above the boiling point of the lubricant (e.g., about 150° C.), and far below the sintering or coalescing temperature of the polymer, generally at about 327° C. in embodiments.

Next, the article, in embodiments, is reheated at a temperature higher than the drying temperature, but below the melt temperature, e.g., above 240° C., in embodiments.

Next, the article is expanded in one or more dimensions. In embodiments where the article is expanded in a single dimension, or in other words “uniaxially expanded,” the process will result in unidirectional elongated fibrils (which are substantially parallel) extending between nodes. These fibrils will have, in embodiments, lengths of about 0.5 microns to about 3 microns. In embodiments, the resulting pores will have approximate lengths of about 3 microns or less, and widths of about 1 microns or less. This pore sizes will not allow for ingrowth, and the article will have good strength. The extent of expansion of the PTFE makes the material softer, and here, that softness makes the ultimate device perform well.

In some embodiments the article is expanded outwardly using tension along an X axis and along a Y axis which is perpendicular to the X axis. In yet other embodiments, numerous other axes. For example, the multiaxial expansion can be created both in X and Y directions as well as two other axes which are angled relative to the X and Y by 45 degrees. Regardless, the process may result in fibril lengths of between about 0.5 microns to about 1.0 microns. In embodiments, the resulting pore size is about less than or equal to 2 microns. Again here, these pore sizes will not allow for ingrowth, and the multiaxial expansion gives great strength. Also with the multiaxial arrangement, the extent of expansion of the PTFE makes the material softer, and here, that softness makes the ultimate device perform well.

Next, after expansion, the article is subjected to a final heat-treating step. In this step, the material is restrained in its expanded state and heated above the thermal transition temperature at about 350° C. to lock the structure in place.

Now expanded and locked, the ePTFE article is allowed to cool over a period of time at a lower temperature, e.g., at ambient.

Once the article has been processed as discussed above, the article can be presented for use along with existing implant systems and/or methods. The article can also be configured for use as a surgical implant.

The article can also be cut into desired sections or otherwise configured in ways making each section able to function as an individual sling.

As an optional additional step, an antimicrobial coating can be applied to the PTFE strip.

### Example 1

In an embodiment, a resin paste was formed by blending 100% PTFE fine powder with an extrusion-aid (e.g., mineral spirits). The resulting resin paste was then formed into an extrusion pellet.

The PTFE article was extruded as a rectangular cross section, and calendered to a thinner cross section while wet. The ultimate thickness was about 0.60 mm after calendering.

After calendering, the lubricant was removed by subjecting the article to heat at a temperature of about 150° C. in order to dry the article (remove the mineral spirits).

With the lubricant now removed, the process moved on to an expansion step. In this embodiment of the expansion step, the article was then reheated at a temperature above 240° C. but below the melt temperature and expanded uniaxially. This expansion was made by securing each end of the article and expanding the article in a single direction longitudinally.

Following expansion, the material was restrained in its expanded state and heated above the thermal transition temperature at about 350° C. to lock the structure in place.

Now expanded, the ePTFE article was allowed to cool over a period of time at a lower temperature, e.g., at ambient.

The extent of expansion of the PTFE was shown to give the material softness and other desired parameters discussed above making it ideal for use as a surgical sling. The article has fibril lengths which are essentially less than about 3 micron. This size inhibits cells or bacteria from penetrating the material. Another noteworthy property is that the fibrils occupy limited space in the article, whereas the node sizes are relatively much larger. About 80% of the area is comprised of solid nodes.

### Example 2

In this embodiment, the same processes were followed as expressed above in Example 1 except that after calendering and lubricant removal, the article was multiaxially expanded. More specifically, the article was expanded in two dimensions (multiaxially), e.g., longitudinally and laterally (in directions offset by 90 degrees).

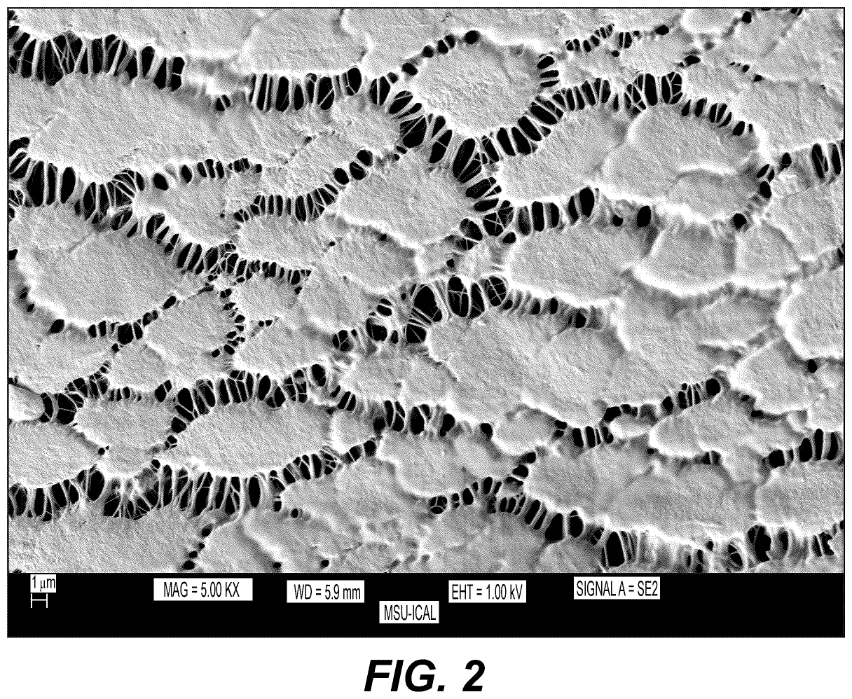

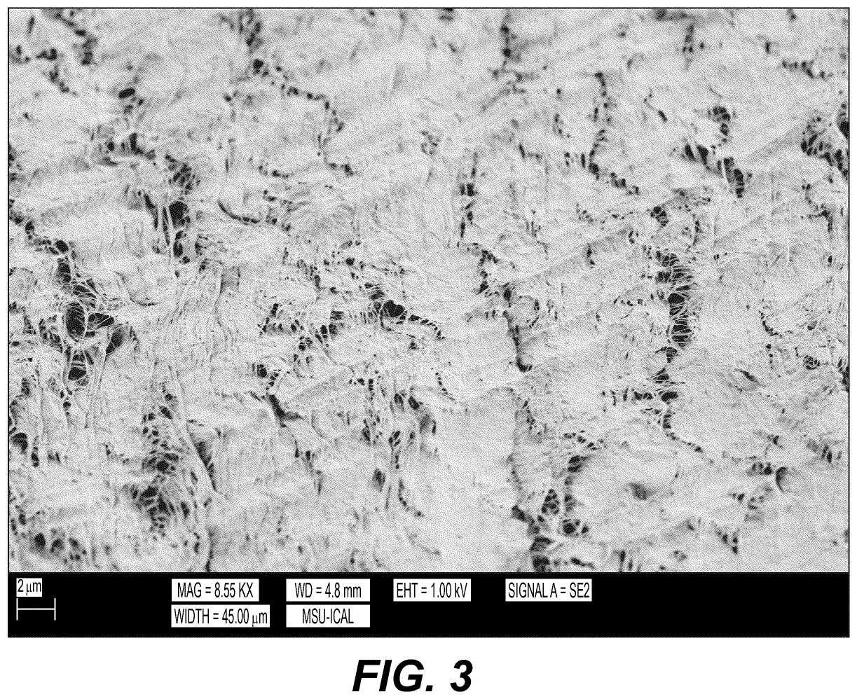

Again here, the expansion of the PTFE was shown to give the material softness and other desired parameters making it ideal for use as a surgical sling. FIG. 2 is a micrograph taken of the material manufactured according to the processes discussed in this example and FIG. 3 is a cross section of what is shown in FIG. 2. Referring to the figure, it can be seen that the article has fibril lengths which, generally speaking, range from about 0.5 micron to about 1.0 micron. The uniaxial expansion creates elongated pores between the nodes which have widths that range in size but are always lower than 2 micron. This size inhibits cells or bacteria from penetrating the material. Again here, fibrils occupy limited space in the article, and the node sizes are relatively larger. As can be seen from the FIGS. 2 and 3 micrographs, more than about 80% of the area is comprised of solid nodes.

### Anchors

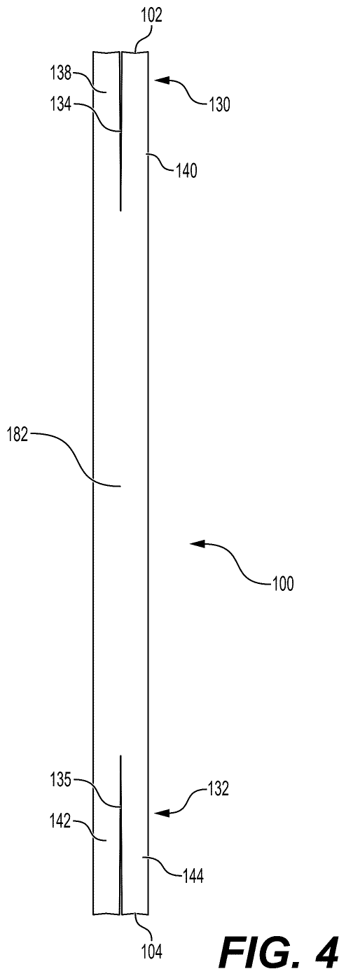

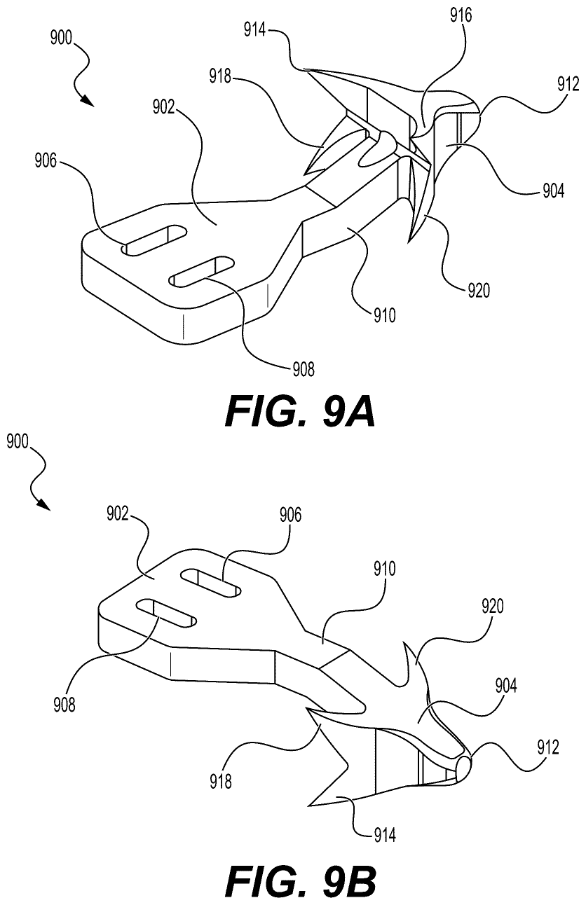

Once the ePTFE material, as discussed above, has been produced, it is cut into strips (or into any configuration, as would be understood by those skilled in the art and as is needed given the application) and the ends are configured for attachment to body internal structures (e.g., muscle, etc.). A first embodiment of how this can be accomplished is shown in FIGS. 4-8B and a second embodiment is shown in FIGS. 9A-15. More specifically, the ends of the cut strips (e.g., ends 102 and 104) are adapted to receive anchors 150 and 152 in the first embodiment and two anchors as represented by anchor 900 in the second embodiment.

FIG. 4 shows a first side 182 of article 100. Both sides are identical. The article 100 has a first end 102 and second end 104 which each have been configured to establish connection locations 130 and 132. In embodiments, connection location 130 has been modified to include a slit 134 and opposing connection location 132 similarly includes a slit 135. Slit 134 is made lengthwise starting at the approximate center of the end 102 of the article 100 and separates the article 100 into independent parallel left and right portions 138 and 140 at the connection location 130. Slit 135 is made lengthwise through the approximate center of article 100 and begins at end 104, separating article 100 into independent left and right portions 142 and 144 at connection location 132. The inwardly made slits 134 and 135 are cut into the article 100 a distance from each of ends 102 and 104 respectively so that portions 138, 140 and 142, 144 may be used to secure anchors 150 and 152 in the first embodiment, or two anchors as represented by anchor 900 in the second embodiment, as will be discussed hereinafter.

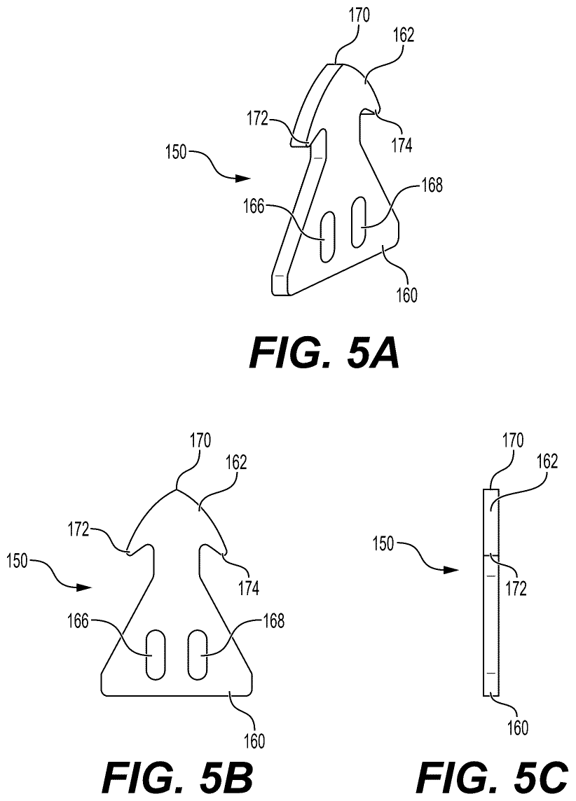

With reference to FIGS. 5A, 5B, and 5C the details of anchor 150 are shown, however it should be noted that anchor 152 is substantially the same as (identical to in some embodiments) anchor 150 and shares similar components. In embodiments, the anchors 150 and 152 are formed from a relatively rigid substantially solid full density PTFE material. Each anchor 150 and 152 includes a base portion 160 and a head portion 162. In embodiments, base portion 160 has a triangular shape which converges upward towards a head portion 162. The base portion 160 includes apertures 166 and 168 which will participate in connection to article 100. The apertures 166 and 168, in embodiments, are configured parallel to one another and can have an ovular or rectangular shape. Above the apertures 166 and 168, the head portion 162 extends away from the base portion 160.

In embodiments, head portion 162 includes a pointed tip 170 and downwardly and outwardly projecting wings 172 and 174 which terminate in points, all of which will be used for securement into bodily tissues. The pointed tip 170, which leads insertion upon an implant procedure, points ahead of the base portion 160 and can be sharp to pierce a bodily tissue encountered, e.g., muscle. Because the wings 172 and 174 project backward during an insertion into tissue, they will secure an implanted head portion 162 against extraction after implant.

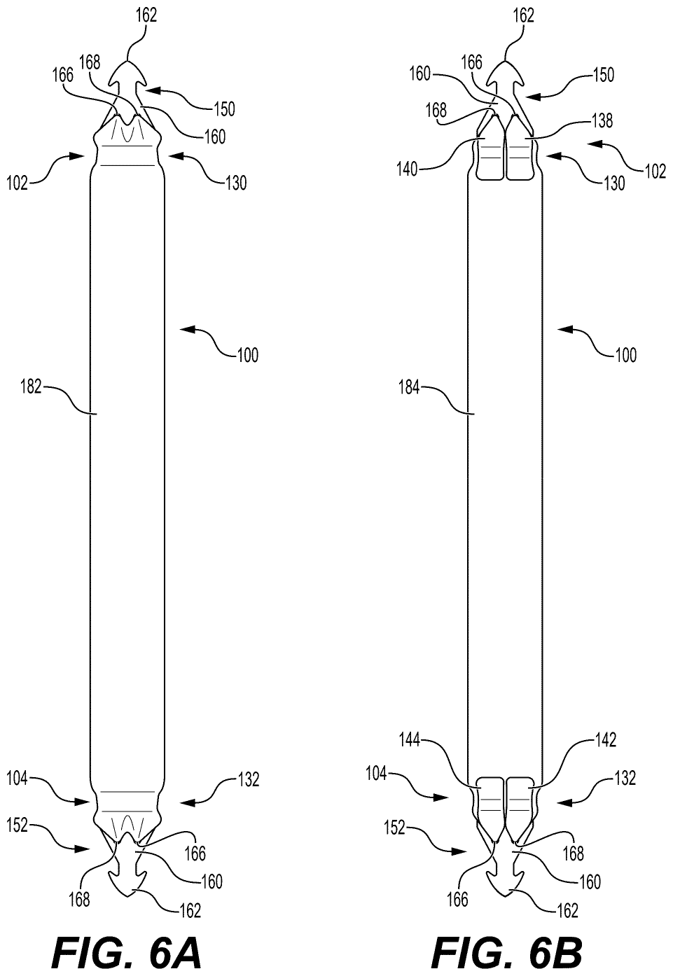

FIGS. 6A-8B show the PTFE article 100 attached to each of the anchors 150 and 152. The joinder utilizes the slits 134 and 135 at connection locations 130 and 132. More specifically, portions 138, 140, 142, and 144 at each end of article 100 are configured to be tucked through the apertures 166 and 168 for each of anchors 150 and 152. More specifically, the portions 138 and 140 formed by slit 134 at connection location 130 secure the anchor 150 to the end 102 of article 100, and the portions 142 and 144 formed by slit 135 at connection location 132 secure the anchor 152 to the end 104 of article 100. The apertures 166 and 168 on each of anchors 150 and 152 are sized and shaped to allow portions 138, 140, 142, and 144 to be tucked through a corresponding aperture 166 or 168 on either of anchors 150 or 152. When tucked through either of apertures 166 or 168 on side 182 of the article 100 shown in FIG. 6A, the portions 138, 140, 142, and 144 come through and are heat treated to adhere to the other side 184 of article 100 as seen in FIG. 6B to permanently secure the anchors 150 and 152 at each end.

In terms of specifics, portion 138 may be tucked through the aperture 166 and the portion 140 may be tucked through the aperture 168 of the anchor 150 to be folded down over back side 184 as shown in FIG. 6B. Additionally, the portion 142 may be tucked through the aperture 166 and the portion 144 may be tucked through the aperture 168 of the anchor 152 to be received onto backside 184. In embodiments, after the portions 138, 140, 142, and 144 have been tucked through a corresponding aperture 166 or 168 on either of anchors 150 or 152 the tucked through portion may be adhered or melted to the backside 184 of article 100 (FIG. 6B).

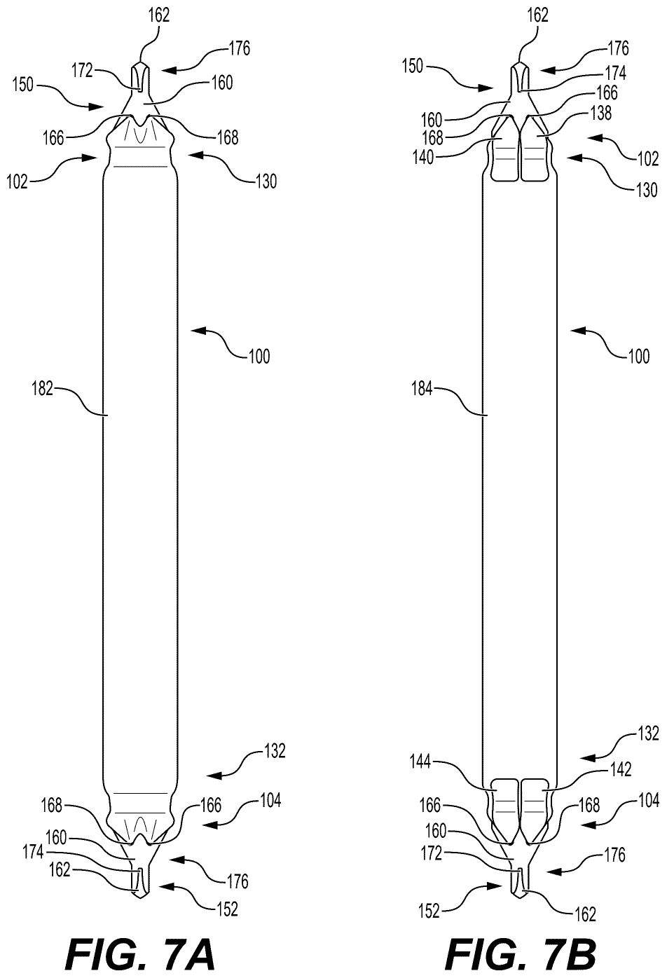

FIGS. 6A and 7A show a first side 182 when article 100 is attached to the anchors 150 and 152. Portion 138 is tucked and pulled through the aperture 166 and portion 140 is tucked and pulled through aperture 168 of anchor 150 such that the anchor 150 is substantially inserted completely through slit 134. FIGS. 6B and 7B, show a back side 184 of article 100 wherein the portions 138 and 140 are then heat fused to the anchors 150 and 152 and the band 100. The portions 138 and 140 (when pulled through apertures 166 and 168) are folded towards the second side 184 of the anchor 150. The folding of the tucked through lengths of portions 138 and 140 towards the second side 184 of article 100 substantially locks the anchor 150 in place at the crease of each portion 138 and 140 after heat treatment. Anchor 152 is similarly attached using the portions 142 and 144 pulled through the apertures 166 and 168 and folded towards second side 184.

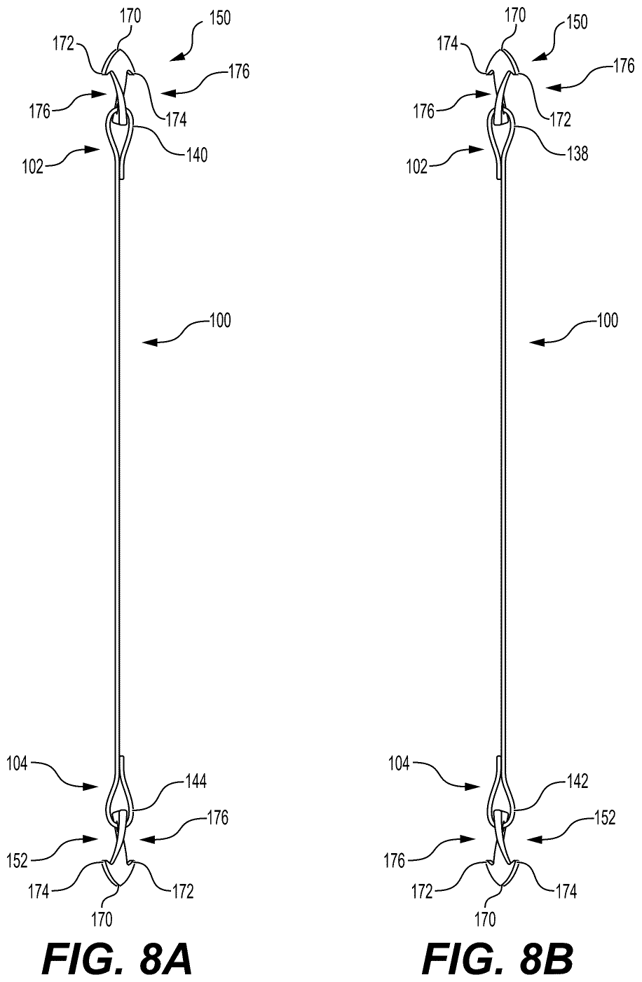

With reference to FIGS. 7A, 7B, 8A, and 8B, the anchors 150 and 152 can include a bend 176 which, in embodiments, positions the head portion 162 approximately perpendicular to the base portion 160. In some embodiments, the bend 176 may be approximately a ninety-degree angle relative to the base portion 160 and article 100. The bend 176 can be formed before or after the anchors 150 and 152 are attached to the article 100. In embodiments, the bend 176 is formed by securing each base 160, heating the portions of each head 162 above each base 160, and then rotating each tip ninety degrees relative to a secured base 160.

The ninety-degree bend 176 created in each instance allows for the catching of backwardly projecting wings 172 and 174 to more thoroughly be secured after pointed tip 170 has penetrated into a tissue or bodily structure. With reference to FIG. 7A, the bend 176 substantially directs the head portion 162 perpendicular to article 100 and out of the page. The twist created in the tips 170 and wings 172 and 174 can also be seen in the side views shown in FIGS. 8A and 8B.

In alternative embodiments, the anchors could be left as they appear in FIGS. 6A and 6B where the tips are not twisted, but remain in the same plane as the body of each anchor. Thus, the surgeon could implant the FIGS. 6A and 6B embodiment as shown.

FIGS. 8A and 8B show a side view of the portions 138 and 140 pulled through the apertures 166 and 168 and reveals the length of the portions 138, 140, 142, and 144 pulled through apertures 166 and 168. In embodiments, the length of portions 138 and 140 pulled through apertures 166 and 168 can be adhered or melted to the second side 184 of the anchors 150 and 152. It is recognized that other bonding or adhesion methods may be used in combination with or in place of heat bonding.

Those skilled in the art will recognize that numerous different sorts of ways that the anchors 150 and 152 are connected to ends 102 and 104 exist in the art, e.g., the ends can be: (i) secured into clamps existing on each anchor 150 and 152; (ii) ends 102 and 104 can be apertured for receipt of snaps on each anchor 150 and 152; (iii) the ends of the article 100 can be knotted and then secured into V-shaped grooves formed into each anchor 150 and 152; (iv) the anchors 150 and 152 can be attached using sutures; (v) attached using trocars; or (vi) other methods.

Turning now to FIGS. 9A, 9B, and 10-15, a second embodiment of an anchor 900 for use with an article as described herein is shown. Those skilled in the art will appreciate that anchor 900 can replace anchors 150, 152 for use with article 100 using the teachings describe above. Specifically, article 100 can secure to two anchors (each being replicas of anchor 900) at connection locations 130, 132. Those skilled in the art will also appreciate that any teachings and features for any anchor embodiment can be used to modify other embodiments herein.

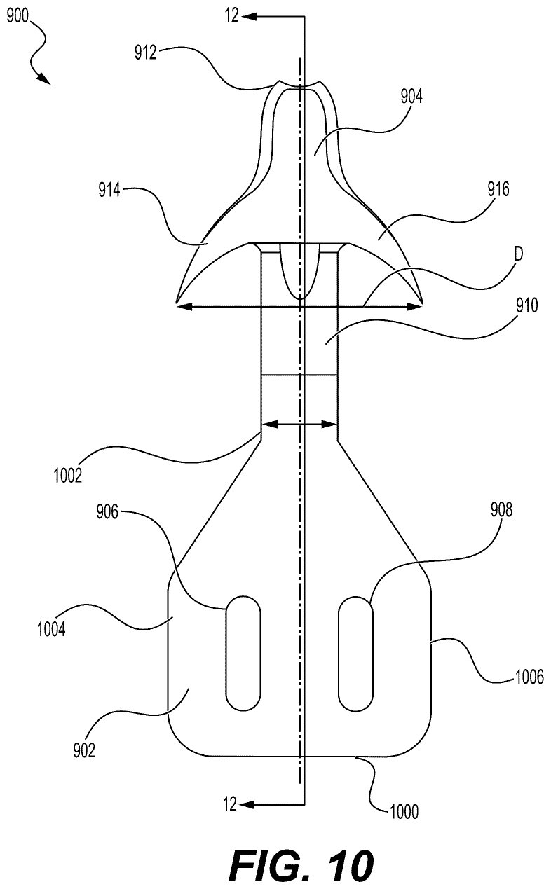

Anchor 900 includes a base portion 902 and a head portion 904 being connected, and in embodiments, being integrally connected and formed from a relatively rigid substantially solid full density polytetrafluoroethylene (PTFE) material. As best shown in FIG. 10, in this embodiment, base portion 902 extends from a bottom end 1000 to a top end 1002, wherein two side edges 1004, 1006 extend upward from bottom end 1000 and run parallel for a distance until converging inward to extend to top end 1002. The base portion 902 includes apertures 906 and 908 which will participate in connection to article 100, as previously described. The apertures 906 and 908, in embodiments, are configured parallel to one another and can have an ovular or rectangular shape. The apertures 906 and 908 are positioned within the base portion 902 in the area wherein the side edges 1004, 1006 run parallel to one another and extend in a direction upward from base portion 902.

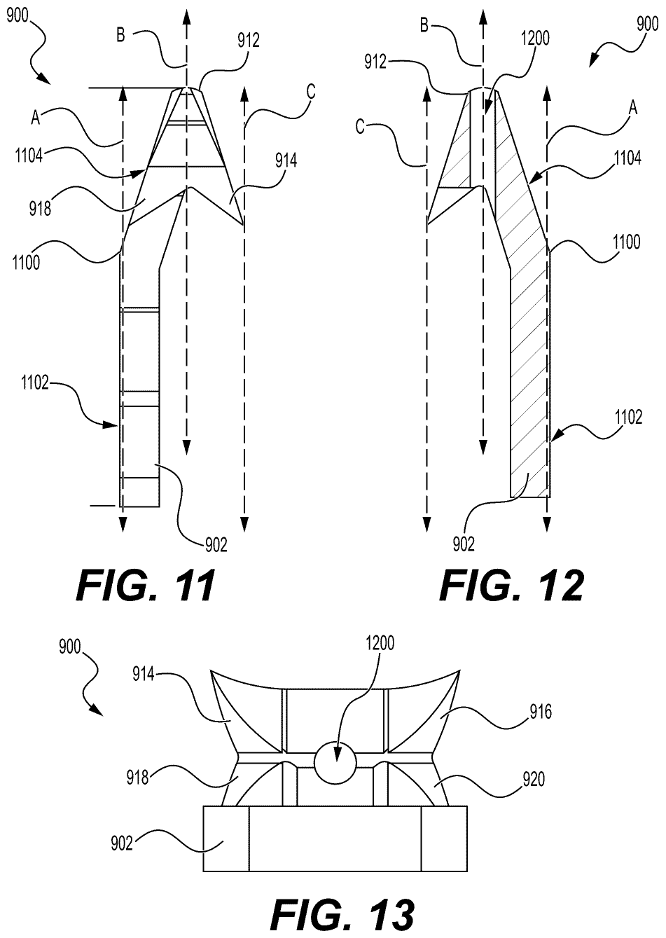

Anchor 900 further includes a connecting portion 910 that connects base portion 902 and head portion 904 together. Connecting portion 910 is substantially equal in width, and in embodiments equal to, the width of top end 1002 of base portion 902. Connecting portion 910 is best shown in FIGS. 11 and 12, wherein a bend 1100 extends within connecting portion 910. Bend 1100 creates an angle between base portion 902 and head portion 904 such that head portion 904 extends outward at said angle that is less than 180 degrees as shown. In the embodiment shown, this angle is greater than 90 degrees.

Turning back to FIGS. 9A and 9B, head portion 904 includes a substantially pointed tip 912 which allows for insertion into bodily tissue. Head portion 904 further includes four outwardly and downwardly pointed wings 914, 916, 918, 920. Specifically, head portion 904 flares down and back from tip 912 such that two wings 914, 916 are projected down and out within a first plane, and two wings 918, 920 are projected down and out within a second plane. Each wing 914, 916, 918, 920 ends at a tipped point, and accordingly tip 912 allows for insertion into bodily tissue, while wings 914, 916, 918, 920 prevent easy removal after insertion, thereby retaining the anchor within the bodily tissue after insertion.

As shown best in FIG. 10, wings 914, 916, 918, 920 project outward to a distance, as shown with line D, that is substantially equal to a width of bottom end 1000 of base portion 902. Those skilled in the art will appreciate that exact dimensions may vary.

Turning again to FIGS. 11 and 12, because of bend 1100, the tip 912 of head portion 904 is positioned in a plane offset from a plane of base portion 902. In other words, when viewed from a side angle (as shown in FIG. 11) a back surface 1102 of base portion 902 and a back surface 1104 of head portion 904 are bent at an angle relative to one another, the angle being less than 180 degrees as shown. Accordingly, back surface 1102 extends within a first plane (Plane A), tip 912 of the head portion 904 is positioned within a second plane (Plane B) that is offset from the first plane, and finally wings 914, 916 are projected outward and downward to align within a third plane (Plane C) that is offset again from the first and second plane. Wings 918, 920 project downward and backward from tip 912, though the tips of these wings 918, 920 align with, or fall inside of plane A. The side angle view depicted in FIG. 11 best demonstrates the arrow shape created between wing 914, tip 912, and wing 918. The same arrow shape is created on the opposite side of anchor 900 between wing 916, tip 912, and wing 920.

As shown in FIGS. 12 and 13, an elongated opening 1200 extends through head portion 904. In embodiments, this elongated opening 1200 is tubular shaped and extends completely such that both ends of elongated opening 1200 are open. Elongated opening 1200 allows for use of anchor 900 with an insertion tool (not shown), wherein said insertion tool is configured to extend through elongated opening 1200 and protrude therefrom, the insertion tool being used to dissect the bodily tissue and guide placement of the article. Once anchor 900 is deployed, the insertion tool is resected out of the tissue.

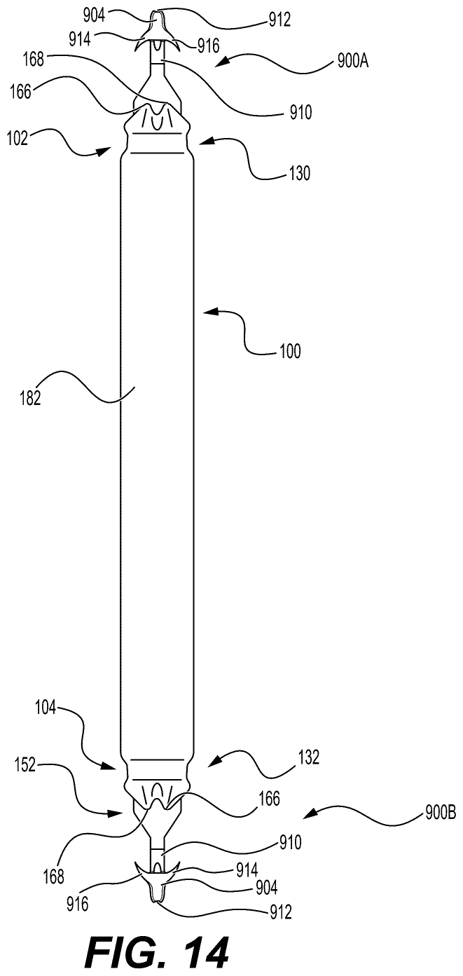

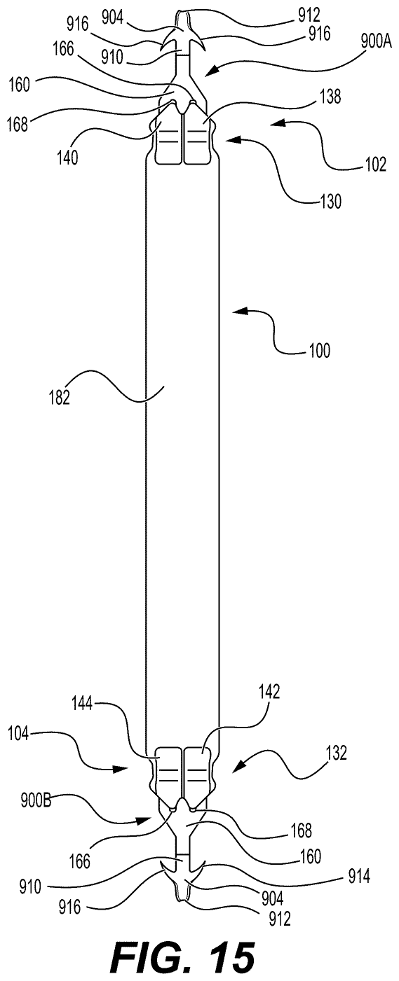

Anchor 900 attaches to article 100 (from FIG. 4) as shown in FIGS. 14 and 15. Specifically, the joinder utilizes the slits 134 and 135 at connection locations 130 and 132. More specifically, portions 138, 140, 142, and 144 at each end of article 100 are configured to be tucked through the apertures of two anchors, each identical to anchor 900, at connection locations 130, 132. More specifically, the portions 138 and 140 formed by slit 134 at connection location 130 secure one anchor 900A to the end 102 of article 100, and the portions 142 and 144 formed by slit 135 at connection location 132 secure a second anchor 900B to the end 104 of article 100. The apertures 166 and 168 on each of the two anchors 900A, 900B are sized and shaped to allow portions 138, 140, 142, and 144 to be tucked through a corresponding aperture 166 or 168 on each anchor. When tucked through either of apertures 166 or 168 on side 182 of the article 100, the portions 138, 140, 142, 144 come through and can be heat treated to adhere to the other side 184 of article 100 to permanently secure the anchors at each end, as shown in FIG. 15. Accordingly, anchor 900 (represented by anchors 900A and 900B in FIGS. 14 and 15) is adhered to article 100 such that the overall construction functions as a surgical implant system.

The pore sizes existing in the expanded supporting band, e.g., between 1-3 microns, are small enough that bacteria is excluded from the article. At the same time, the article created does not resist adhesion to tissue and adheres to a desirable extent. This is accomplished using the relatively small pore sizes disclosed (less than 3 microns) along with a resulting relatively soft biocompatible surface presented by the ePTFE material.

Although the descriptions above relate to the use of ePTFE articles as prosthetic slings to stabilize a patient's urethra or bladder as described above, they could also be useful in providing support for other organs. Additionally, multiple strips could be used together for certain applications. Further, the articles could be used for the support of rectal or pelvic muscles in other applications.

Many different arrangements of the various components depicted, as well as components not shown, are possible without departing from the spirit and scope of what is claimed herein. Embodiments have been described with the intent to be illustrative rather than restrictive. Alternative embodiments will become apparent to those skilled in the art that do not depart from what is disclosed. A skilled artisan may develop alternative means of implementing the aforementioned improvements without departing from what is claimed.

It will be understood that certain features and sub-combinations are of utility and may be employed without reference to other features and sub-combinations and are contemplated within the scope of the claims. Not all steps listed in the various figures need be carried out in the specific order described.

1. An anchor for receiving an end of an article configured for use as a surgical implant, the anchor comprising:
a base portion having one or more apertures configured to receive the end of the article such that the article is secured to the anchor; and
a head portion attached to the base portion and extending therefrom, the head portion having a tip and at least two wings extending downward from the tip such that each of the at least two wings end in a wing tip;
wherein the head portion is configured for insertion through bodily tissue via the tip and is configured to be retained within the bodily tissue via the at least two wings; and
wherein the base portion is configured to retain the article to the bodily tissue.

2. The anchor of claim 1, wherein the base portion further comprises a bottom end, a top end, and two side edges extending upward from the bottom end and running parallel for a first distance and converging inward for a second distance until reaching the top end.

3. The anchor of claim 1, wherein the base portion and the head portion are integrally constructed from polytetrafluoroethylene (PTFE) material.

4. The anchor of claim 1, further comprising a bend positioned between the base portion and the head portion, wherein bend positions the at least two wings into a substantially perpendicular arrangement relative to the base portion.

5. The anchor of claim 1, wherein the at least two wings further comprise a first pair of wings each having a wing tip and a second pair of wings each having a wing tip.

6. The anchor of claim 5, further comprising a connecting portion connecting the base portion and the head portion, the connecting portion including a bend configured such that a back surface of the head portion extends away from a back surface of the base portion at an angle of less than 180 degrees.

7. The anchor of claim 6, wherein the back surface of the base portion extends within a first plane; the tip is positioned within a second plane, the second plane being parallel and offset from the first plane; each wing tip of the first pair of wings is positioned within a third plane, the third plane being parallel and offset from both of the first plane and the second plane; and each wing tip of the second pair of wings is positioned between the first plane and the second plane.

8. The anchor of claim 1, further comprising an elongated opening extending through the head portion.

9. A surgical implant system, comprising:
an article configured to support a bodily tissue, the article having:
a body extending from a first end forming a first connection location to a second end forming a second connection location, the body formed of a biocompatible, bacteria-resistant fluoropolymer material;
wherein the biocompatible, bacteria-resistant fluoropolymer material comprises a plurality of nodes and a plurality of fibrils, the plurality of fibrils interconnecting the plurality of nodes to form a plurality of pores; and
wherein the biocompatible, bacteria-resistant fluoropolymer material comprises a microporous structure which is relatively closed to ingrowth and bacterial penetration; and
an anchor configured to secure to the article and retain the article to the bodily tissue, the anchor having:
a base portion having one or more apertures configured to connect to the first connection location of the article such that the article is secured to the anchor; and
a head portion attached to the base portion and extending therefrom, the head portion having a tip and at least two wings extending downward from the tip such that each of the at least two wings end in a wing tip;
wherein the head portion is configured for insertion through the bodily tissue via the tip and is configured to be retained within the bodily tissue via the at least two wings; and
wherein the base portion is configured to retain the article to the bodily tissue.

10. The surgical implant system of claim 9, wherein the at least two wings further comprise a first pair of wings each having a wing tip and a second pair of wings each having a wing tip.

11. The surgical implant system of claim 10, wherein the anchor further comprises a connecting portion connecting the base portion and the head portion, the connecting portion including a bend configured such that a back surface of the head portion extends away from a back surface of the base portion at an angle of less than 180 degrees.

12. The surgical implant system of claim 11, wherein the back surface of the base portion extends within a first plane; the tip is positioned within a second plane, the second plane being parallel and offset from the first plane; each wing tip of the first pair of wings is positioned within a third plane, the third plane being parallel and offset from both of the first plane and the second plane; and each wing tip of the second pair of wings is positioned between the first plane and the second plane.

13. The surgical implant system of claim 9, wherein the anchor further comprises an elongated opening extending through the head portion.

14. The surgical implant system of claim 9, wherein the biocompatible, bacteria-resistant fluoropolymer material comprises expanded Polytetrafluoroethylene (PTFE).

15. The surgical implant system of claim 9, wherein the body is multiaxially expanded such that the plurality of fibrils radiate between the plurality of nodes to define the plurality of pores, and wherein the plurality of fibrils are about 0.5 microns to about 1.0 microns in length and the plurality of pores are less than or equal to 2.0 microns in size.

16. The surgical implant system of claim 9, wherein the body is uniaxially expanded such that the plurality of fibrils radiate between the plurality of nodes to define the plurality of pores, and wherein the plurality of fibrils are about 0.5 to about 3.0 microns in length and the plurality of pores are less than or equal to 3.0 microns by 1.0 microns in size.

17. The surgical implant system of claim 9, wherein the body has a thickness of about 0.60 mm.

18. The surgical implant system of claim 9, wherein the article is configured for use as a pubovaginal sling.

19. The surgical implant system of claim 9, wherein the plurality of fibrils multiaxially radiate between the plurality of nodes to form the plurality of pores and wherein the plurality of pores are less than or equal to 2 microns.

20. The surgical implant system of claim 9, wherein the plurality of fibrils expand substantially uniaxially between the plurality of nodes to form the plurality of pores, wherein the plurality of pores are less than or equal to 3.0 microns by 1.0 microns.
